(19) United States
(12) Patent Application Publication
(10) Pub. No.: US 2004/0231682 A1
(43) Pub. Date: Nov. 25, 2004

(54) Scanned small spot ablation with a high-rep-rate

(76) Inventors: Richard Stoltz, Plano, TX (US); Jeff Bullington, Chuluota, FL (US)

Correspondence Address: CHALKER FLORES, LLP, 12700 PARK CENTRAL, STE. 455, DALLAS, TX, 75251, US

(21) Appl. No.: 10/849,585

(22) Filed: May 19, 2004

Related U.S. Application Data
(60) Provisional application No. 60/471,972, filed on May 20, 2003.
(60) Provisional application No. 60/503,578, filed on Sep. 17, 2003.

(51) Int. Cl. A61B 19/00; A61B 18/20

(52) U.S. Cl. 128/898; 606/2

(57) ABSTRACT

The present invention is a system and method of ablation laser-machining, that includes the steps of generating pulses at 1 to 50 MHz by one or more semiconductor-chip laser diodes, each pulse having a pulse-duration less than three picoseconds, directing a less than 1 square mm beam of the pulses to a work-piece with an ablating pulse-energy-density; and scanning the beam with a power-driven scanner to ablate a scanned area at least 25 times larger than the beam area.

### TECHNICAL FIELD OF THE INVENTION

The present invention relates in general to the field of light amplification and, more particularly, to scanned small spot ablation with a high-repetition-rate.

### BACKGROUND OF THE INVENTION

Ablation type laser-machining can be done rapidly while avoiding collateral damage, by using a scanned small spot with a very-high rep-rate (e.g., 10 MHz) and operating within a narrow range of energy densities. This can achieve ablation level energy density (e.g., 0.1 to 10 Joules/square centimeter) over a reasonably large (e.g., 5 mm diameter) area with a single semiconductor optical amplifier (SOA) putting out a few micro-Joules per pulse.

The use of a high-repetition rate allows the small scanned spot to ablate the larger area rapidly, while, generally avoiding unevenly ablated regions within the area and collateral damage. For example, in surgical applications, a scanned 20 micron spot can be scanned by a mirror and a pair of piezoelectric actuators. For example, a one micro-Joule pulse from a SOA, might give about 0.5 Joules/square centimeter into the optical delivery system and deliver 0.25 Joules/square centimeter to the surface being ablated. A 20 MHz rep rate spot could be linearly scanned to give a line of overlapping spots, and a surgeon could move the line for area coverage. A second linear scanner could transversely scanned the line to give an adjustable width. The length and width of the line could be adjusted by the surgeon. The repetition rate could also be raised or lowered to adjust the removal rate. Further, a train of pulses can allows a quasi-CW (continuous wave) operation that improves system efficiency, e.g., lessening the number of current up-ramps and down-ramps. The train of pulses is generated by one or more semiconductor-chip diodes, due both to their high efficiency and their good performance at high repetition rates

Laser ablation is efficiently done with a beam of short pulses (generally a pulse-duration of three picoseconds or less). Laser machining can remove ablatively material by disassociate the surface atoms and melting the material. Techniques for generating these ultra-short pulses (USP) are described, e.g., in a book entitled “Femtosecond Laser Pulses” (C. Rulliere—editor), published 1998, Springer-Verlag Berlin Heidelberg New York. Generally large systems, such as Ti:Sapphire are used for generating ultra-short pulses.

USP phenomenon was first observed in the 1970's, when it was discovered that mode-locking a broad-spectrum laser could produce USP's. The minimum pulse duration attainable is limited by the bandwidth of the gain medium, which is inversely proportional to this minimal or Fourier-transform-limited pulse duration. Mode-locked pulses are typically very short and will spread (i.e., undergo temporal dispersion) as they traverse any medium. Subsequent pulse-compression techniques are often used to obtain USP's. Pulse dispersion can occur within the laser cavity so that compression techniques are sometimes added intra-cavity. When high-power pulses are desired, they are intentionally lengthened before amplification to avoid internal component optical damage. This is referred to as “Chirped Pulse Amplification” (CPA). The pulse is subsequently compressed to obtain a high peak power (pulse-energy amplification and pulse-duration compression).

### SUMMARY OF THE INVENTION

It has been found that ablation-type laser-machining can be done rapidly while avoiding collateral damage, by using a scanned small spot with a very-high rep-rate (e.g., 10 MHz), e.g., by operating within a narrow range of energy densities. Ablation-type laser-machining can be achieve using an ablation level energy density (e.g., 0.1 to 10 Joules/square centimeter) over a reasonably large (e.g., 5 mm diameter) area with a single semiconductor optical amplifier (SOA) emitting a few micro-Joules per pulse. The use of a high-rep-rate allows the small scanned spot to ablate the larger area rapidly, while generally avoiding unevenly ablated regions within the area and collateral damage.

For example, in surgical applications a scanned 20 micron spot can be scanned by a mirror and a pair of piezoelectric actuators. The scanned small spot can be, e.g., a one micro-Joule pulse from a SOA, which might give about 0.5 Joules/square centimeter into the optical delivery system and deliver 0.25 Joules/square centimeter to the surface being ablated. A 20 MHz repetition rate spot could be linearly scanned to give a line of overlapping spots, and a surgeon could move the line for area coverage. A second linear scanner could transversely scanned the line to give an adjustable width. The length and width of the line could be adjusted by the surgeon. The repetition rate could also be raised or lowered to adjust the overall removal rate. Further, a train of pulses can allows a quasi-continuous wave (CW) operation that improves system efficiency, e.g., lessening the number of current up-ramps and down-ramps. Preferably, the train of pulses is generated by one or more semiconductor-chip diodes, due both to their high efficiency and their good performance at high repetition rates.

One embodiment of the present invention uses a scanned small spot ablation with a high-repetition-rate for light amplification. In one embodiment, the method of the present invention uses a small spot scan with a high repetition rate between about one and about ten MHz and operating within a narrow range of energy densities allows rapid ablation-type laser-machining while avoiding collateral damage. One embodiment achieves ablation level energy density of between about 0.1 and about 10 Joules/square centimeter over a reasonably large about 5 mm diameter area with a single semiconductor optical amplifier (SOA) putting out 10 micro-Joules per pulse or less. The use of a high-rep-rate allows the small scanned spot to ablate a larger area rapidly, while generally avoiding unevenly ablated regions within the area and collateral damage.

For example, in surgical applications, a scanned 20 micron spot can be scanned by a mirror and a pair of piezoelectric actuators. For example, a 50 micro-Joule pulse from a SOA, could give about 16 Joules/square centimeter into the optical delivery system and deliver 8 Joules/square centimeter to the surface being ablated. A 20 MHz repetition rate spot could be linearly scanned to give a line of overlapping spots, and a surgeon could move the line for area coverage. A second linear scanner could transversely scanned the line to give an adjustable width using the same mirror or a second mirror. The length and width of the line could be adjusted by the surgeon. The repetition rate could also be raised or lowered by the surgeon to adjust the removal rate.

When the ablation is part of a surgical procedure, the ablating pulse-energy-density is between 1 and 10 times the ablation threshold, and more preferably about 1 to 3 times the ablation threshold to minimize collateral damage.

The present invention uses a method of ablation laser-machining, comprising: generating 1 to 50 MHz pulses by one or more semiconductor-chip laser diodes, each pulse having a pulse-duration less than three picoseconds; directing a less than onesquare mm beam of the pulses to a work-piece with an ablating pulse-energy-density; and scanning the beam with a power-driven scanner to ablate a scanned area at least 25 times larger than the beam area.

In one embodiment, the pulse-energy-density is 0.1 to 20 Joules/square centimeter, the beam area is 1 to 2,500 square microns and/or the scanned area at least 100 times larger than the beam area. Preferably, the pulse-duration is 50 femtoseconds to 1 picosecond and/or the pulse-energy-density is between 0.1 and 8 Joules/square centimeter on the work-piece. The pulses may be generated at 1 to 50 MHz. The beam is scanned in at least one, and preferably two or more directions, and can be scanned in a spiral. The beam is preferably scanned to travel at a speed of at least one meter per second on the work-piece.

The present invention also includes a method of ablation laser-machining, including generating 0.6 to 100 MHz pulses, each pulse having a pulse-duration less than three picoseconds; directing a less than one square mm beam of the pulses to a work-piece with an ablating pulse-energy-density; and scanning the beam with a power-driven scanner over a scanned area at least 25 times larger than the beam area. The ablation may be part of a surgical procedure.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Laser machining is most efficiently conducted with a beam of very short pulses (generally a pulse-duration of three picoseconds or less) in a controlled range of energy density (generally about 0.1 to 20 Joules/square centimeter, and preferably 0.1 to 8 Joules/square centimeter). Lasers can remove e.g., a slit of material 500 microns wide, or a circle, or a rectangle. The amount of material that needs to be removed is greatly reduced by the small (0.001 up to about 1 mm) spot size, which reduces the required power and allows machining with smaller and less expensive lasers (including portable semiconductor-chip-diode systems).

Further, due to the small diameter of the laser beam, relative motion (e.g., vibration) between the laser beam and the work-piece can prevent successive pulses from overlapping properly and movement such as vibration can cause uneven ablation. Note that uses such as surgical procedures can use surface ablation or cutting, and can use overlapping ablation to produce a cut surface. In all such uses, a train of pulses is preferably generated by one or more semiconductor-chip diodes. The train of pulses allows a quasi-CW operation that improves system efficiency, e.g., lessening the number of current up-ramps and down-ramps. A cutting-line of laser-produced ablation (including in the circumference a circle of ablation to cut out a large hole) can be produced. There are, however, applications where a single laser-produced hole completely penetrating a work-piece is desired. The very high repetition-rates greatly reduce interference from vibration or other undesired motion.

In one embodiment the scanning can be accomplished by the use of a small piezoelectric driven mirror. This scanning element can be small and fit in a dry erase pen size device and dither the focal spot across a larger spot such as a two (2) millimeter diameter region. A two (2) mm region can be identified, e.g., by a visible light source such as a red LED (Light Emitting Diode) imaged on the surface of the biomaterial. The scanning mirror can have two operational modes. One mode is where the ablating light is scanned across the entire two (2) mm diameter region making a circular cut. The second mode is when the cut is made in a two (2) mm long, 100 μm wide stripe. The initial device can use a reasonably long focal length imaging element to permit a reasonable working distance. The beam is scanned across the tissue and removing the material in a nearly painless manner with virtually no residual damage, but the cut region may have bleeding since the ultra short pulse (USP) does not cauterize the region. In the case of an unacceptable level of bleed is induced in the removal region a second laser diode ˜1 W QCW GaAs laser can be used to cauterize the region. Since the USP laser and the cauterizing laser operate at approximately the same wavelength they can use the same optical beam train and imaging system, including drive steering mirror. The cauterizing laser may be triggered manually.

For example, one micron may be removed per 20 micron diameter pulse, and thus ˜3×10⁻⁷ cu-mm removed per shot. At 10 MHz, this is three (3) cubic mm/second or 180 cubic mm/min removed. At 10 MHz, in 0.001 seconds 10,000 pulses could be swept (e.g., by a piezoelectric actuator) over, e.g., 0.5 cm, and the spacing between pulse centers would be about 0.5 microns. With the 20-micron spot diameter, the centerline would see 40 pulses, and a 40 micron deep groove in a single pass. For cutting, this could give a 40 mm deep cut, five (5) mm long in One (1) second. For removal of an area, if a single-spot-wide line were used and scanned at One (1) kHz, and even if the surgeon moved the line at one (1) cm/second the line would retrace each area for four (4) times and the groove would be ˜0.16 mm deep. Rather than let the line retrace during area removal, each of the next 100 lines might be automatically stepped over 10 microns by a second (e.g., piezoelectric) actuator, to give a ˜one (1) 1 mm thick line (or 500 lines might be stepped over ten microns, to give a ˜five mm thick line).

The linear scanning could be done with a rotating disc with a number (e.g., 20) of flat mirror faces around the periphery. This can give the beam an angular scan. At 3,600 rpm (60 rps) 20 mirrors would give a 1.2 kHz scan repetition rate. A second rotating disc at a lower rpm could add line thickness. Changing line length (or width) can be done with a pair of adjustable reflectors. The scanning may also be accomplished with a piezoelectric (e.g., quartz crystals) or magnetostrictive block with a mirrored face at a shallow angle to the input beam. The scanning can provide beams controllably, parallel-displaced by an electrical signal across the crystal.

Additional mirrors at shallow angles could be added to the light path to increase the displacement, e.g., by 50×. The block can be 5 cm long and have a 10⁻⁶ cm/cm displacement and get a 50× increase (2.5×10⁻⁴ cm displacement) due to its shallow angle with the beam. If a 0.5 cm displacement is desired an additional 1,000× is needed, and e.g., two additional mirrors give less than 50× each could be used (if all angles gave 46.4× it would be enough). If all angles gave 20×, three additional mirrors would be enough.

Magnetostriction can give a sufficiently large maximum displacement. Iron-60% cobalt gives 70×10⁻⁶ cm/cm at a field of around H-450. Iron gives 4×10⁻⁶ cm/cm, at a field of around H-50 and may be more practical in some applications due to the lower required field.

Alternately, one or two piezoelectric blocks may be used. Note that a pair of actuators could be mounted to move a single mirror to give parallel offset lines in one direction and an angular movement at right angles. Preferably, a scan over the area is completed in less than 100 milliseconds, and more preferably in less than 10 milliseconds.

It should be noted that this method works especially well with semiconductor-chip diodes. Semiconductor-chip diodes can have high efficiency (e.g., about 50%) and have short energy-storage-lifetimes (e.g., a few nanoseconds). With a small, e.g., 20 micron spot, the ablating energy can be furnished by a single semiconductor optical amplifier (SOA) putting out less than 10 micro-Joules per pulse (low energy density also limits collateral damage). The other types of lasers (e.g., a Ti:sapphire amplifier pumped by a Nd:YAG laser, which is in turn pumped by flash-lamps or pump diodes) generally have energy-storage-lifetimes (e.g., in the hundreds of microsecond range), which is convenient for accumulating energy and releasing it in a short period of time as a high-energy pulse. The Ti:sapphire/Nd:YAG-type lasers have generally been used for generating short, high energy pulses, but the efficiencies are very low (generally less than 1%) and the pulse energies drop off rapidly when operated at high repetition rates (when they begin to heat up, and when time between pulses becomes short and starts to reduce the time for accumulating energy for the next pulse). Conversely, semiconductor optical amplifiers can provide a microsecond long train of pulses of nearly constant energy with nanosecond spacings. Thus, while other types of lasers could be used, semiconductor-chip diodes are preferred. Note however, that fiber amplifiers, especially when operated at high repetition rates, or solid-state optical amplifiers that can be directly pumped by pump diodes (e.g., Cr:YAG amplifiers) may also be used.

For example, a 100 femtosecond pulse can be time-stretched to make an optical pulse signal ramp (of, e.g., increasing, wavelength) which is amplified (at comparatively low instantaneous power), and time-compressed into an amplified 100 femtosecond pulse. Generally, a series of pulses are generated, and thus a series of wavelength-ramps are used (e.g., a “saw-tooth” waveform with 50 “teeth” may be amplified by the SOAs without turning the current off between the teeth). Thus, although the amplifiers are amplifying continuously during the 50-tooth waveform, the time-compression will separate the optical output into 50 separate pulses.

Additionally, the SOAs have an energy storage lifetime on the order of a few nanoseconds. The nanosecond energy storage lifetime allows the stretch pulses to be amplified effectively and have constant energy per pulse and achieve maximum repetition rates above 50 MHz. Repetition rates above about 100 MHz would see the decrease in the energy per pulse as most solid-state lasers do at repetition rates >1 KHz. Another benefit to the SOAs is the ability to use conventional thermal management schemes and off-the-shelf drive circuitry with a moderate average power requirement and high efficiencies. Once the stretched pulse is amplified, the optical pulses are then recompressed giving a high intensity pulse with a pulse width in the femtosecond regime. The compression can be accomplished by using a dispersive element that acts as a spectral filter, thereby delaying one end of the spectrum so that the spectrum is compressed into a very narrow temporal slot. If one stretches a pulse to 20 ns, amplifies it and then recompresses it to a 200-fs pulse width, the final amplification peak power is reduced by a factor of 10⁵, without decreasing final pulse power. The longer pulse and lower amplitude drive current combine to reduce the thermal spikes in the quantum well to a few degrees Celsius and dramatically reduces the resistive losses at the contact.

Further, a train of pulses allows a quasi-CW operation that improves system efficiency, e.g., lessening the number of power up-ramps and down-ramps. The train of pulses may be generated by one or more semiconductor-chip diodes, due both to their high efficiency and their good performance at high repetition rates.

Directing a beam of the pulses to a work-piece with a pulse-energy-density of 0.1 to 20 Joules/square centimeter can produce ablation of the work-piece surface. In some embodiments, a 0.05 to 1 microsecond-long train of pulses is used. The pulse-duration can be 50 femtoseconds to 1 picoseconds, and the pulses at intervals are 1 to 10 nanoseconds. The pulse-energy-density may be between 0.1 and 8 Joules/square centimeter on the work-piece.

Semiconductor laser diodes are preferred for generating the ultra-short pulses. Semiconductor laser diodes typically are of III-V compounds (composed of one or more elements from the third column of the periodic table and one or more elements from the fifth column of the periodic table, e.g., GsAs, AlGaAs, InP, InGaAs, or InGaAsP). Other materials, such as II-VI compounds, e.g., ZnSe, can also be used. Typically lasers are made up of layers of different III-V compounds (generally, the core layer has higher index of refraction than the cladding layers to generally confine the light to a core). Semiconductor lasers have been described, see e.g., “Femtosecond Laser Pulses” (C. Rulliere—editor), 1998, Springer-Verlag, New York (Chapter 5) relevant portions incorporated herein by reference. It should be noted that the method of the present invention works well with semiconductor-chip diodes. Semiconductor-chip diodes can have high efficiency (e.g., about 50%) and have short energy-storage-lifetimes (e.g., a few nanoseconds), attributes not generally available in other types of lasers.

Information of such a system and other information on ablation systems are given in co-pending provisional applications listed in the following paragraphs (which are also at least partially co-owned by, or exclusively licensed to, the owners hereof) and are hereby incorporated by reference herein (provisional applications listed by docket number, title and United States Provisional Patent Application Serial Number):

Docket number ABI-1 “Laser Machining”—provisional application United States Provisional Patent Applications, Ser. No. 60/471,922; ABI-4 “Camera Containing Medical Tool” United States Provisional Patent Applications, Ser. No. 60/472,071; ABI-6 “Scanned Small Spot Ablation With A High-Rep-Rate” United States Provisional Patent Applications, Ser. No. 60/471,972; and ABI-7 “Stretched Optical Pulse Amplification and Compression”, United States Provisional Patent Applications, Ser. No. 60/471,971, were filed May 20, 2003;

ABI-8 “Controlling Repetition Rate Of Fiber Amplifier” United States Provisional Patent Applications, Ser. No. 60/494,102; ABI-9 “Controlling Pulse Energy Of A Fiber Amplifier By Controlling Pump Diode Current” United States Provisional Patent Applications, Ser. No. 60/494,275; ABI-10 “Pulse Energy Adjustment For Changes In Ablation Spot Size” U.S. Provisional Patent Applications, Ser. No. 60/494,274; ABI-11 “Ablative Material Removal With A Preset Removal Rate or Volume or Depth” U.S. Provisional Patent Applications, Ser. No. 60/494,273; ABI-12“Fiber Amplifier With A Time Between Pulses Of A Fraction Of The Storage Lifetime”; ABI-13 “Man-Portable Optical Ablation System” U.S. Provisional Patent Applications, Ser. No. 60/494,321; ABI-14 “Controlling Temperature Of A Fiber Amplifier By Controlling Pump Diode Current” U.S. Provisional Patent Applications, Ser. No. 60/494,322; ABI-15 “Altering The Emission Of An Ablation Beam for Safety or Control” U.S. Provisional Patent Applications, Ser. No. 60/494,267; ABI-16 “Enabling Or Blocking The Emission Of An Ablation Beam Based On Color Of Target Area” U.S. Provisional Patent Applications, Ser. No. 60/494,172; ABI-17 “Remotely-Controlled Ablation of Surfaces” U.S. Provisional Patent Applications, Ser. No. 60/494,276 and ABI-18 “Ablation Of A Custom Shaped Area” United States Provisional Patent Applications, Ser. No. 60/494,180; were filed Aug. 11, 2003. ABI-19 “High-Power-Optical-Amplifier Using A Number Of Spaced, Thin Slabs” United States Provisional Patent Applications, Ser. No. 60/497,404 was filed Aug. 22, 2003;

Co-owned ABI-20 “Spiral-Laser On-A-Disc”, United States Provisional Patent Applications, Ser. No. 60/502,879; and partially co-owned ABI-21 “Laser Beam Propagation in Air”, United States Provisional Patent Applications, Ser. No. 60/502,886 were filed on Sep. 12, 2003. ABI-22 “Active Optical Compressor” United States Provisional Patent Applications, Ser. No. 60/503,659 and ABI-23 “Controlling Optically-Pumped Optical Pulse Amplifiers” United States Provisional Patent Applications, Ser. No. 60/503,578 were both filed Sep. 17, 2003;

ABI-24 “High Power SuperMode Laser Amplifier” United States Provisional Patent Applications, Ser. No. 60/505,968 was filed Sep. 25, 2003, ABI-25 “Semiconductor Manufacturing Using Optical Ablation” United States Provisional Patent Applications, Ser. No. 60/508,136 was filed Oct. 2, 2003, ABI-26 “Composite Cutting With Optical Ablation Technique” United States Provisional Patent Applications, Ser. No. 60/510,855 was filed Oct. 14, 2003 and ABI-27 “Material Composition Analysis Using Optical Ablation”, United States Provisional Patent Applications, Ser. No. 60/512,807 was filed Oct. 20, 2003;

ABI-28 “Quasi-Continuous Current in Optical Pulse Amplifier Systems” U.S. Provisional Patent Applications, Ser. No. 60/529,425 and ABI-29 “Optical Pulse Stretching and Compressing” U.S. Provisional Patent Applications, Ser. No. 60/529,443, were both filed Dec. 12, 2003;

ABI-30 “Start-up Timing for Optical Ablation System” U.S. Provisional Patent Applications, Ser. No. 60/539,026; ABI-31 “High-Frequency Ring Oscillator”, U.S. Provisional Patent Applications, Ser. No. 60/539,024; and ABI-32 “Amplifying of High Energy Laser Pulses”, U.S. Provisional Patent Applications, Ser. No. 60/539,025; were filed Jan. 23, 2004; and

ABI-33 “Semiconductor-Type Processing for Solid-State Lasers”, U.S. Provisional Patent Applications, Ser. No. 60/543,086, was filed Feb. 9, 2004; and ABI-34 “Pulse Streaming of Optically-Pumped Amplifiers”, United States Provisional Patent Applications, Ser. No. 60/546,065, was filed Feb. 18, 2004. ABI-35 “Pumping of Optically-Pumped Amplifiers”, was filed Feb. 26, 2004.

The examples used herein are to be viewed as illustrations rather than restrictions, and the invention is intended to be limited only by the claims. For example, the invention applies to other semiconductor materials such as II-VI compounds. In some embodiments, an InP laser diode generates light within a III-V semiconductor structure at a wavelength of about 1550 nm out a surface of the semiconductor structure.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of ablation laser-machining, comprising:
generating pulses at 1 to 50 MHz by one or more semiconductor-chip laser diodes, each pulse having a pulse-duration less than three picoseconds;
directing a less than 1 square mm beam of the pulses to a work-piece with an ablating pulse-energy-density; and
scanning the beam with a power-driven scanner to ablate a scanned area at least 25 times larger than the beam area.

2. The method of claim 1, wherein the pulse-energy-density is 0.1 to 20 Joules/square centimeter.

3. The method of claim 1, wherein scanned area at least 100 times larger than the beam area.

4. The method of claim 1, wherein the pulse-duration is 50 femtoseconds to 1 picosecond.

5. The method of claim 1, wherein beam area is 1 to 2,500 square microns.

6. The method of claim 1, wherein the pulse-energy-density is between 0.1 and 8 Joules/square centimeter on the work-piece.

7. The method of claim 1, wherein the pulses are generated at 0.1 to 50 MHz.

8. The method of claim 1, wherein the beam is scanned in one direction.

9. The method of claim 1, wherein the beam is scanned in two directions.

10. The method of claim 1, wherein the beam is scanned in a spiral.

11. A method of ablation laser-machining, comprising:
generating 0.6 to 100 MHz pulses, each pulse having a pulse-duration less than three picoseconds;
directing a less than 1 square mm beam of the pulses to a work-piece with an ablating pulse-energy-density; and
scanning the beam with a power-driven scanner over a scanned area at least 25 times larger than the beam area.

12. The method of claim 11, wherein the ablation is part of a surgical procedure.

13. The method of claim 11, wherein the ablation is part of a surgical procedure, and the ablating pulse-energy-density is between 1 and 10 times the ablation threshold.

14. The method of claim 11, wherein the ablation is part of a surgical procedure, and the ablating pulse-energy-density is between 1 and 3 times the ablation threshold.

15. The method of claim 11, wherein the pulses are generated by at least one optical amplifier.

16. The method of claim 11, wherein the pulses are generated by one semiconductor optical amplifier (SOA) and the pulses contain less than about 50 micro-Joules per pulse.

17. The method of claim 11, wherein the pulses are generated by one fiber amplifier and the pulses contain less than 10 micro-Joules per pulse.

18. The method of claim 11, wherein the beam is rasterized.
